(19) United States
(12) Patent Application Publication
(10) Pub. No.: US 2013/0131153 A1
(43) Pub. Date: May 23, 2013

(54) CULLIN 5 AS A REGULATOR OF HSP90 CLIENTS: A NEW TARGET FOR DRUG DEVELOPMENT

(71) Applicants: Xiao-Fang Yu, Ellicott City, MD (US); Elana S. Ehrlich, Baltimore, MD (US); Rongzhen Xu, Hangzhou (CN)

(72) Inventors: Xiao-Fang Yu, Ellicott City, MD (US); Elana S. Ehrlich, Baltimore, MD (US); Rongzhen Xu, Hangzhou (CN)

(73) Assignee: THE JOHNS HOPKINS UNIVERSITY, Baltimore, MD (US)

(21) Appl. No.: 13/510,093

(22) PCT Filed: Nov. 16, 2010
(86) PCT No.: PCT/US10/56811

§ 371 Date: Jan. 28, 2013

Related U.S. Application Data
(60) Provisional application No. 61/261,559, filed on Nov. 16, 2009.

(51) Int. Cl. G01N 33/573; C12Q 1/68

(52) U.S. Cl. CPC G01N 33/573; C12Q 1/68
USPC 514/44 R; 435/6.18; 435/7.4; 435/375

(57) ABSTRACT

Cullin5 (Cul5) E3 ubiquitin ligase is shown to be linked with the heat shock protein 90 (Hsp90) chaperone complex. Hsp90 participates in the folding of its client proteins into their functional conformation. Many Hsp90 clients have been reported to be aberrantly expressed in a number of cancers. Cul5 is shown to interact with members of the Hsp90 chaperone complex as well as the Hsp90 client, ErbB2, Cul5 is recruited to the site of ErbB2 at the plasma membrane and subsequent induction of polyubiquitination and proteasomal degradation. Cul5 is also involved in the regulation of another Hsp90 client, Hif-1α Cul5 degradation of ErbB2 occurs independently of ElonginB-ElonginC function. The involvement of Cul5 in Hsp90 client regulation has implications in the effectiveness of Hsp90 targeted chemotherapy, which is currently undergoing clinical trials. The link between Cul5 and Hsp90 client regulation may represent an avenue for cancer drug development.

### CROSS-REFERENCE TO RELATED APPLICATIONS

This application claims the benefit of U.S. Provisional Application No. 61/261,559, filed Nov. 16, 2009; which is incorporated herein by reference in its entirety.

### STATEMENT OF GOVERNMENTAL INTEREST

This invention was made with U.S. government support under grant no. A1062644. The U.S. government has certain rights in the invention.

### FIELD OF THE INVENTION

The present invention relates to the field of molecular biology.

### BACKGROUND OF THE INVENTION

The regulation of client proteins by heat shock protein 90 (Hsp90) plays an important role in critical cellular processes such as cell cycle control and apoptosis. Dysregulation is linked to cancer and neurological diseases (1, 2). Hsp90 is a molecular chaperone responsible for the correct folding of proteins, allowing them to attain their proper functional conformations (3). Many client proteins of Hsp90 are overexpressed oncogenes that are critical for the transformed phenotype observed in tumors (4-6). Clients of Hsp90 are also regulated by the ubiquitin/proteasome system (7). However, the identity of the cellular E3 ubiquitin ligase(s) that regulate(s) Hsp90 client proteins is still elusive.

The Cullin family of RING E3 ubiquitin ligases are modular enzymes that act as a scaffolding to bring a specific substrate within close proximity to the E2 ubiquitin conjugating enzyme, thereby facilitating ubiquitination and subsequent proteasomal degradation (8, 9). There are seven known human Cullin proteins, Cul1, 2, 3, 4a, 4b, 5, and 7, with diverse functions from cell cycle regulation, to DNA repair, to regulation of developmental processes. Substrate specificity is determined by the combinatorial nature of E3 ligase assembly. In the case of Cul1, diversity is achieved by the assembly of different F-box substrate receptor proteins with Cul1 through a single Skp1 adaptor protein (10). Each F-box protein determines the specificity of Cul1 substrates. Cul3 is unique in having one protein with two domains, one functioning as an adaptor forming an interface with Cullin, and the other acting as a substrate receptor (9).

Cul2 and 5 are interesting in that they both use ElonginB-C adaptor proteins, through which they bind a SOCS box containing substrate receptor (8). Whether a substrate receptor recruits Cul2 or Cul5 depends on an additional Collin binding interface. In the case of cellular substrate receptors, Cullin selection is determined by the presence of a VHL or SOCS box motif (11), in the case of HIV and SIV, a zinc-stabilized helix mediates Cul5 selection (12-15).

### SUMMARY OF THE INVENTION

The present invention is based, in part, on the seminal discovery that Cul5 regulates Hsp90 clients. As shown herein, Cul5 interacts with the Hsp90 chaperone complex and the Hsp90 client-ErbB2, and Cul5 is recruited to the site of ErbB2 on the plasma membrane, thereby inducing its polyubiquitination and proteasome-mediated degradation. Other Hsp90 client proteins, such as HIF1-α, were also regulated by Cul5. Cul5-mediated degradation of ErbB2 was observed to occur in the absence of the traditional Cul5 adaptors ElonginB and ElonginC, suggesting that a component of the Hsp90 chaperone complex may serve this function. This is an example of a link between Hsp90 and the Cullin family of E3 ubiquitin ligases. This is also a report of an ElonginB-ElonginC-independent Cul5 E3 ubiquitin ligase.

Hsp90 is a well-established target for chemotherapy (16). The results shown herein provide an explanation for the decrease or loss of Cul5 expression that is observed in a number of cancers (17). The data also suggest that Cul5 levels could potentially influence susceptibility to certain cancers and the effectiveness of anti-cancer treatment with geldanamycin or its derivatives, which are currently in human clinical trials.

Accordingly, in certain aspects, the present invention provides methods for determining whether an agent causes an increase in the expression of Cullin 5 protein. In certain embodiments, the method comprises the steps of (a) contacting the agent with a eukaryotic cell under conditions which, in the absence of the agent, permit expression of Cullin 5 protein; (b) determining, after a suitable period of time, the amount of expression of Cullin 5 protein in the cell; and (c) comparing the amount of expression determined in step (b) with the amount of expression which occurs in the absence of the agent, whereby an increased amount of expression in the presence of the agent indicates that the agent causes an increase in the expression of Cullin 5 protein.

In one embodiment, determining the amount of expression of Cullin 5 protein in the cell may be performed by determining the amount of Cullin 5 protein-encoding mRNA present in the cell. In an alternative embodiment, determining the amount of expression of Cullin 5 protein in the cell may be performed by determining the amount of Cullin 5 protein present in the cell. In a specific embodiment, the determining the amount of Cullin 5 protein in the cell is performed using an antibody specific for such protein.

In other aspects, the present invention provides methods for determining whether an agent causes an increase in the activity of Cullin 5 protein. In particular embodiments, the method comprises the steps of (a) contacting the agent with a eukaryotic cell under conditions which, in the absence of the agent, permit activity of Cullin 5 protein; (b) determining, after a suitable period of time, the amount of activity of Cullin 5 protein in the cell; and (c) comparing the amount of activity determined in step (b) with the amount of activity of Cullin 5 protein which occurs in the absence of the agent, whereby an increased amount of activity in the presence of the agent indicates that the agent causes an increase in the activity of Cullin 5 protein.

In particular embodiments, the eukaryotic cell is present in a cell culture. The cell may be a tumor cell.

In other aspect, the present invention provides methods for increasing the expression of Cullin 5 in a eukaryotic cell. In one embodiment, the method comprises introducing into the cell an agent which specifically increases the expression of Cullin 5 protein in the cell. The agent may be a polynucleotide. More specifically, the polynucleotide may be an expression vector encoding Cullin 5. In particular embodiments, the call may be a tumor cell. Moreover, the cell may be characterized by the overexpression of an HSP90 client protein.

The present invention further provides methods for treating a subject afflicted with an HSP90 client protein related disorder. In a specific embodiment, the method comprises administering to the subject a therapeutically effective amount of an agent which specifically increases the expression of Cullin 5 protein in the cells of the subject. The method may further comprise the step of administering an HSP90 inhibitor to the subject. In certain embodiments, the subject is human. The agent may be a polynucleotide. In particular embodiments, the polynucleotide may be an expression vector encoding Cullin 5.

The HSP client protein may be selected from the group consisting of ErbB2, Hif-1α, c-Rav-1, Akt/PKB, CDK4, Polo-1 kinase, Met, mutant p53, and hTERT. In a specific embodiment, the HSP client protein is ErbB2. In an alternative embodiments, the HSP client protein is Hif-1α.

### BRIEF DESCRIPTION OF THE FIGURES

FIG. 1 demonstrates that Cul5 interacts with the Hsp90-Hsp70 chaperone complex. In FIG. 1A, Cul5-HA and Cul5-cmyc were immunoprecipitated with anti-HA-conjugated agarose beads. The eluates were analyzed by SDS-PAGE and Coomassie staining, and bands were identified by mass spectrometry. In FIGS. 1B and 1C, Cul5-HA and Cul5-cmyc were immunoprecipitated with anti-HA-conjugated agarose beads in (B) 293T and (C) U87 cells. Eluates were analyzed by SDS-PAGE and Western blotting with antibodies against HA, Hsp90, and Hsp70. In FIG. 1D, 293T cell lysates were incubated with antibody against Hsp70 or IgG and protein A/G agarose. Protein complexes were immunoprecipitated and analyzed by SDS-PAGE and Western blotting with antibodies against Hsp70 and Cul5. In FIG. 1E, 293T cells were treated with GA or DMSO as indicated. Protein complexes were immunoprecipitated with antibodies against Cul5 and protein A/G agarose. Eluates were analyzed by SDS-PAGE and Western blotting with antibodies against Cul5, Hsp90, and Hsp70. In FIG. 1F, 293T cells were transfected with the ErbB2 expression vector and treated with GA or MG132 as indicated. Cell lysate were incubated with antibody against ErbB2 and with protein A/G agarose. Immuno-precipitates were analyzed by SDS-PAGE, followed by Western blotting with antibody against Cul5.

FIG. 2 shows that Cul5 is recruited to ErbB2 at the plasma membrane. In FIGS. 2A-2F, 293T cells were transfected with the ErbB2 expression vector (2D-2F) or empty vector control (2A-2C). Twenty-four hours after transfection, the cells were plated on glass coverslips and incubated for 16 h. Cells were fixed, permeablized, and stained with antibodies against endogenous Cul5 and ErbB2. Slides were visualized using a Zeiss Meta 510 confocal microscope and viewed with LSM software. Images are equivalent slices of a z-stack.

FIG. 3 reveals that Cul5 is required for polyubiquitination and proteasomal degradation of ErbB2. In FIG. 3A, CHIP knockdown does not completely stabilize ErbB2. 293T cells were transfected with ErbB2 and either CHIP or control siRNA as indicated. At 48 h after transfection, the cells were treated with GA or DMSO for 16 h. ErbB2 stability was assessed by Western blotting with antibodies against ErbB2, CHIP, and ribosomal p19. In FIG. 3B, ErbB2 degradation is inhibited by the Cul5 dominant-negative mutant. 293T cells were transfected with ErbB2 and Cul5ΔNedd8-cmyc where indicated. At 48 h after transfection, the cells were treated with GA or DMSO for 16 h as indicated. ErbB2 stability was assessed by Western blotting with antibodies against ErbB2, cmyc, and ribosomal p19. In FIG. 3C, Cul5 shRNA inhibits GA-mediated degradation of ErbB2 in SKBR3 cells. SKBR3 cells were infected with lentiviruses containing GFP targeting or Cul5 targeting shRNA. Twenty-four hours post infection, cells were selected with puromycin for 1 week. Cells were treated with GA or equivalent volume of DMSO for 2 h. Cells were harvested and analyzed by Western blot against ErbB2, Cul5, or β-actin where indicated. In FIG. 3D, a Cul5 dominant-negative mutant is shown to inhibit ErbB2 polyubiquitination. 293T-cells were transfected with ErbB2 and Cul5ΔNedd8-cmyc as indicated. At 48 h after transfection, the cells were treated with 5 μM MG132 and 3 μM GA or DMSO for 4 has indicated. Cell lysates were incubated with anti-ErbB2 and protein G-conjugated agarose. Immunoprecipitates were then analyzed by Western blotting with antibodies against ErbB2, myc, and ubiquitin.

FIG. 4 demonstrates that Cul5 is required for ErbB2 and Hif-α degradation. In FIGS. 4A and 4B, Cul5 but not Cul2 is required for GA-mediated ErbB2 degradation. 293T′ cells were transfected with the ErbB2 expression vector and Cul5, Cul2, or control siRNA as indicated. Forty-eight hours post transfection, the cells were treated with GA or DMSO for 16 h. ErbB2 stability was assessed by Western blotting with antibodies against ErbB2, Cul2, Cul5, and ribosomal p19. In FIGS. 4C and 4D, Cul5 is required for GA-mediated degradation of HIF-1α. 293T cells were cotransfected with HIF-1α-HA expression vector and Cul5 or control siRNA or Cul5ΔNedd8 as indicated. At 48 h after transfection, the cells were treated with GA or DMSO for 16 h. HIF-1α stability was assessed by Western blotting with antibodies against HA, Cul5, and ribosomal p19.

FIG. 5 demonstrates that Cul5-mediated degradation of ErbB2 occurs via an ElonginB-ElonginC-independent mechanism. In FIG. 5A, ElonginB and ElonginC are not required for GA-mediated degradation of ErbB2. 293T cells were cotransfected with ErbB2 expression vector and ElonginB or control siRNA as indicated. At 48 h after transfection, the cells were treated with GA or DMSO for 16 h. ErbB2 stability was assessed by Western blotting with antibodies against ErbB2, ElonginB, ElonginC, and ribosomal p19. In FIG. 5B, ElonginB and ElonginC are required for HIV Vif-mediated degradation of APOBEC3G (A3G). 293T cells were cotransfected with A3G and Vif expression vectors and ElonginB or control siRNA where indicated. At 48 h after transfection, the cells were harvested, and A3G stability was assessed by Western blotting with antibodies against A3G-HA, ElonginB, ElonginC, and ribosomal p19. In FIG. 5C, the ElonginC dominant negative mutant can no longer interact with the SOCS box containing substrate receptor, yet retains interaction with Cul5. In FIG. 5D, ElonginC is not required for GA-mediated degradation of ErbB2. 293T cells were cotransfected with ErbB2 expression vector and ElonginCΔ4 or control empty vector as indicated. At 48 h after transfection, the cells were treated with GA or DMSO for 16 h. ErbB2 stability was assessed by Western blotting with antibodies against ErbB2, HA-tagged ElonginC Δ4, and ribosomal p19. In FIG. 5E, ElonginC is required for Vif-mediated degradation of A3G. 293T cells were cotransfected with A3G and Vif expression vectors and ElonginCΔ4 or control empty vector where indicated. Forty-eight hours post transfection A3G stability was assessed by Western blotting with antibodies against HA-A3G, HIV Vif, HA-tagged ElonginC Δ4, and ribosomal p19.

FIG. 6 presents a proposed model for Cul5-mediated regulation of Hsp90 client proteins, GA treatment or ATP hydrolysis induces remodeling of the Hsp90 chaperone complex, resulting in recruitment of the Cul5-E3 ubiquitin ligase, followed by polyubiquitination and proteasomal degradation of ErbB2 or other Hsp90 client proteins.

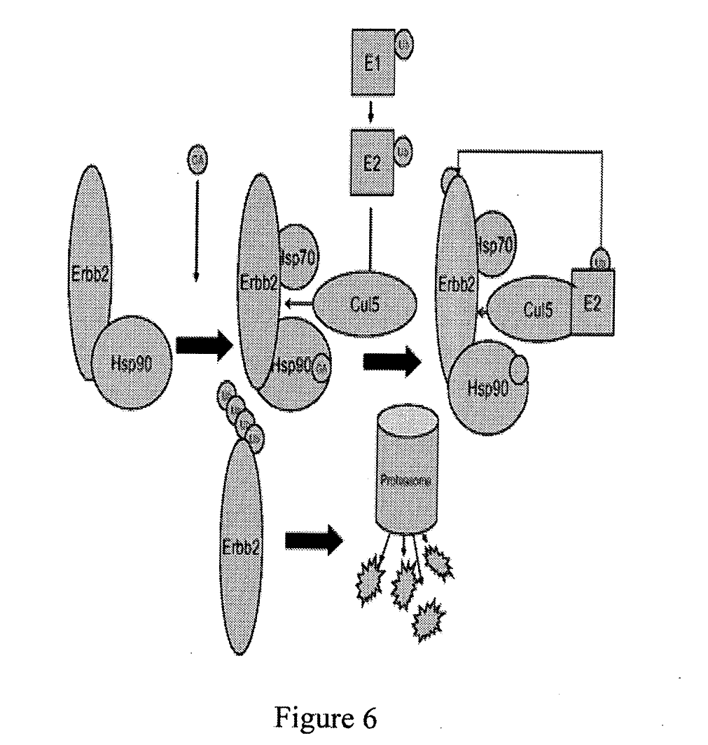

FIG. 7 reveals that Cul5 but not Cul2 co-localizes with ErbB2 at the plasma membrane. In FIGS. 7A and 7B, 293T cells were transfected with the ErbB2 expression vector and Cul2-HA (7A) or Cul5-HA (7B). Twenty-four hours after transfection, the cells were plated on glass coverslips and incubated for 16 h. Cells were fixed, permeablized, and stained with antibodies against HA and ErbB2. Slides were visualized using a Nikon Eclipse 90i upright, microscope and viewed with Velocity software.

FIG. 8 shows that Cul5 co-localizes with ErbB2 at the plasma membrane. In FIGS. 5A and 8B, 293T cells were transfected with the ErbB2 expression vector (8B) or empty vector control (8A). Twenty-four hours after transfection, the cells were plated on glass coverslips and incubated for 16 h. Cells were fixed, permeablized, and stained with antibodies against endogenous Cul5 and ErbB2. Slides were visualized using a Zeiss Meta 510 confocal microscope and viewed with LSM software. Images are sequential slices of a z-stack.

FIG. 9 demonstrates that ErbB2 expression does not drastically affect cell integrity. In FIGS. 9A and 9B, 293T cells were transfected with the Cul5-HA expression vector and ErbB132 (9B) or vector control (9A). Twenty-four hours after transfection, the cells were plated on glass coverslips and incubated for 16 h. Cells were fixed, permeablized, and stained with antibodies against HA and ErbB2. Slides were visualized using a Nikon Eclipse-90i upright microscope and viewed with Velocity software.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Accordingly, in certain aspects, the present invention provides methods for determining whether an agent causes an increase in the expression of Cullin 5 protein. As used herein, the term “agent” means any compound that is being examined for the ability to increase Cullin 5 expression and/or increase Cullin 5 activity. An agent can be any type of molecule including, but not limited to, a polynucleotide, peptide, an antibody, a glycoprotein, a carbohydrate, a small organic molecule, or a peptidomimetic.

The term “polynucleotide” or “nucleic acid” is used broadly herein to mean a sequence of two or more deoxyribonucleotides or ribonucleotides that are linked together by a phosphodiester bond. As such, the term “polynucleotide” includes RNA and DNA, which can be an isolated naturally occurring polynucleotide or portion thereof or a synthetic polynucleotide, and can be single stranded or double stranded, as well as a DNA/RNA hybrid. A polynucleotide agent (or agent) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs. See Pagratis et al., 15 NATURE BIOTECHNOL 68-73 (1997); Jellinek et-al., 34 BIOCHEMISTRY 11363-11372 (1995); and Lin et al., 22 NUCL. ACIDS RES. 5220-34-(1994).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides. See, e.g., Ecker and Crooke, 13. BIOTECHNOLOGY 351-60 (1995); and Tam et al., 22 NUCL. ACIDS RES. 977-86 (1994). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

Peptides also can be useful as agents. The term “peptide” is used broadly herein to refer to a molecule containing two or more amino acids or amino acid analogs (or modified forms thereof) linked by peptide bonds. As such, peptide test agents (or agents) can contain one or more D-amino acids and/or L-amino acids; and/or one or more amino acid analogs, for example, an amino acid that has been derivatized or otherwise modified at its reactive side chain. In addition, one or more peptide bonds in the peptide can be modified, and a reactive group at the amino terminus or the carboxy terminus or both can be modified. Peptides containing D-amino acids, or L-amino acid analogs, or the like, can have improved stability to a protease, an oxidizing agent or other reactive material the peptide may encounter in a biological environment. Further, the stability of a peptide agent (or test agent) can be improved by generating (or linking) a fusion protein comprising the peptide and a second polypeptide (e.g., an Fc domain of an antibody) that increases the half-life of the peptide agent in vivo. Peptides also can be modified to have decreased stability in a biological environment, if desired, such that the period of time the peptide is active in the environment is reduced.

Antibodies provide an example of peptides useful as agents in an assay of the present invention. As used herein, the term “antibody” is used in its broadest sense to include polyclonal and monoclonal antibodies, as well as antigen binding fragments of such antibodies. Antibodies are characterized, in part, in that they specifically bind to an antigen, particularly to one or more epitopes of an antigen. The term “binds specifically” or “specific binding activity” or the like, when used in reference to an antibody, means that an interaction of the antibody and a particular epitope has a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or less. As such, Fab, F(ab′)₂, Fd and Fv fragments of an antibody that retain specific binding activity are included within the definition of an antibody.

The term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains. See Huse et al., 246 SCIENCE 1275-1281 (1989). These and other methods of making, for example, chimeric, humanized, CDR-grated, single chain, and bifunctional antibodies are well known in the art. See generally, Harlow and Lane, ANTIBODIES. A LABORATORY MANUAL (Cold Spring Harbor Laboratory Press, 1999); Borrabeck, Antibody Engineering, 2d ed. (Oxford University Press 1995); Winter and Harris, 14 IMMUNOL TODAY 243-46 (1993); Hilyard et al., PROTEIN ENGINEERING. A PRACTICAL APPROACH (IRL Press 1992); and Ward et al. 341 NATURE 544-46 (1989).

In certain embodiments, the method comprises the steps of (a) contacting the agent with a eukaryotic cell under conditions which, in the absence of the agent, permit expression of Cullin 5 protein; (b) determining, after a suitable period of time, the amount of expression of Cullin 5 protein in the cell; and (c) comparing the amount of expression determined in step (b) with the amount of expression which occurs in the absence of the agent, whereby an increased amount of expression in the presence of the agent indicates that the agent causes an increase in the expression of Cullin 5 protein.

In one embodiment, determining the amount of expression of Cullin 5 protein in the cell may be performed by determining the amount of Cullin 5 protein-encoding mRNA present in the cell. In an alternative embodiment, determining the amount of expression of Cullin 5 protein in the cell may be performed by determining the amount of Cullin 5 protein present in the cell. In a specific embodiment, the determining the amount of Cullin 5 protein in the cell is performed using an antibody specific for such protein. In particular embodiments, the eukaryotic cell is present in a cell culture. The cell may be a tumor cell.

More specifically, as disclosed herein, the methods of the present invention can be performed in vitro (e.g., in a cell free system using purified or partially purified components) or in a cell (e.g., in a cell or tissue culture system). Where the method is performed in vitro, the Cullin 5 may be a purified, naturally occurring Cullin 5 E3 ubiquitin ligase obtained, for example, from an extract or can be a synthetic Cullin 5 prepared, for example, using an in vitro translation or coupled transcription/translation reaction using the known Cullin 5 polynucleotide (GenBank Accession No. X81882). Where the method is performed as a cell based assay, the sample can be a cell sample, wherein Cullin 5 is expressed in the cell.

In other aspects, the present invention provides methods for determining whether an agent causes an increase in the activity of Cullin 5 protein. In particular embodiments, the method comprises the steps of (a) contacting the agent with a eukaryotic cell under conditions which, in the absence of the agent, permit activity of Cullin 5 protein; (b) determining, after a suitable period of time, the amount of activity of Cullin 5 protein in the cell; and (c) comparing the amount of activity determined in step (b) with the amount of activity of Cullin 5 protein which occurs in the absence of the agent, whereby an increased amount of activity in the presence of the agent indicates that the agent causes an increase in the activity of Cullin 5 protein. In particular embodiments, the eukaryotic cell is present in a cell culture. The cell may be a tumor cell.

The activity of Cullin 5 protein may be determined using methods known to those of ordinary skill in the art. In particular embodiments, Cullin 5 activity is determined using a ubiquitin ligase assay. See, e.g., U.S. Pat. No. 7,781,182; U.S. Pat. No. 6,740,495; and U.S. Pat. No. 6,737,244.

The conditions suitable for assaying Cullin 5 activity may be determined using the disclosure presented herein, knowledge of those of ordinary skill in the art, and/or through routine experimentation. Such conditions are exemplified herein, and include, for example, an appropriate concentration of ions sufficient for Cullin 5 activity, as well as appropriate buffer conditions (including pH), salt concentration (e.g., physiological), and other conditions, which can be selected based on whether the assay is performed in a cell free format or is performed in a cell based assay. Agents identified in the methods of the invention can be further evaluated, detected, cloned, sequenced and the like, either in solution or after binding to a solid support.

In other aspect, the present invention provides methods for increasing the expression of Cullin 5 in a eukaryotic cell. In one embodiment, the method comprises introducing into the cell an agent which specifically increases the expression of Cullin 5 protein in the cell. The agent may be a nucleic acid. More specifically, the nucleic acid may be an expression vector encoding Cullin 5. In particular embodiments, the call may be a tumor cell. Moreover, the cell may be characterized by the overexpression of an HSP90 client protein.

The present invention further provides methods for treating a subject afflicted with an HSP90 client protein related disorder. In a specific embodiment, the method comprises administering to the subject a therapeutically effective amount of an agent which specifically increases the expression of Cullin 5 protein in the cells of the subject. The HSP client protein may be selected from the group consisting of ErbB2, Hif-1α, c-Rav-1, Akt/PKB, CDK4, Polo-1 kinase, Met, mutant p53, and hTERT. In a specific embodiment, the HSP client proteins ErbB2. In an alternative embodiments, the HSP client protein is Hif-1α.

In certain embodiments, the subject is human. The agent may be a nucleic acid. In particular embodiments, the nucleic acid may be an expression vector encoding Cullin 5.

The method may further comprise, the step of administering an HSP90 inhibitor to the subject. The Hsp90 inhibitor can be, for example, geldanamycin or a derivative thereof. The Hsp90 inhibitor can also include 17-ally-amino-17-demethoxygeldanamycin (17-AAG), 17-(2-dimethylaminoethyl)amino-17-demethoxygeldanamycin (17-DMAG), tanespimycin, retaspimycin, IPI-493, CNF-1010, alvespimycin, B11B021, SNX-5422 and STAT-9090.

### DISCUSSION

The data presented here suggests that Cul5 is recruited by the Hsp90 complex and serves to regulate Hsp90 client proteins via an ElonginB-ElonginC-independent mechanism. This is an example of a Cullin-RING E3 ubiquitin ligase being involved in chaperone-mediated protein regulation. Cul5 is shown to interact with Hsp90, Hsp70, and ErbB2. Cul5 specifically co-localizes with ErbB2 at the plasma membrane. Cul5 but not Cul2 is required for proteasomal degradation of ErbB2.

Lentivirus delivered shRNA was used against Cul5 to evaluate ErbB2 degradation in SKBR3 breast cancer cells and observed stabilization of ErbB2 in the presence of GA. Increased levels of ErbB2 was also observed in DMSO controls, suggesting that Cul5 plays a role in regulation of ErbB2 in both 293T cells ectopically expressing ErbB2 and ErbB2 positive SKBR3 breast cancer cells in the absence of GA. In addition, Cul5 serves to regulate another Hsp90 client Hif1-α, suggesting a role for Cul5 in Hsp90 client regulation. Interestingly, inhibition of the ElonginB-C module via siRNA and dominant negative mutant suggests that Cul5 may be functioning independently of the traditional adaptor proteins. The ElonginC dominant negative mutant can no longer interact with the SOCS box in both HIV Vif and adenovirus E4orf6, suggesting that it can no longer interact with cellular SOCS box containing substrate receptors. This data suggests that a Cul5 E3 ligase is at work here. It is possible that Cul5 may be interacting directly with the Hsp90/Hsp70 chaperone complex.

Hsp90 client proteins play important roles in numerous cellular processes, including signal transduction, gene regulation, cell cycle control, and apoptosis, and elevated expression of some Hsp90 client proteins has been implicated in the maintenance and progression of a number of cancers. The data shown herein raise the possibility that Cul5 dysregulation may play a role in the overexpression of Hsp90 client proteins in certain tumors. Interestingly, Cul5 expression has been shown to be decreased in a number of cancers, suggesting that Cul5 suppression may be beneficial for tumor development or maintenance (35). The data also suggest that Cul5 can influence the effectiveness of anticancer treatment with GA or its derivatives, and its potential effects should be considered when evaluating the clinical efficacy of these treatments. Thus, the relationship between oncogenesis and Cul5-mediated Hsp90 client regulation may represent an avenue for cancer drug development.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Cells, Plasmids, Transfections, Reagents, and Antibodies.

293T cells [AIDS Research Reagents Program, Division of AIDS, National Institute of Allergy and Infectious Diseases (NIAID), National Institutes of Health (NIH), cat no. 3522] were maintained in DMEM (Invitrogen) with 10% FBS and gentamycin (5 μg/mL; D-10 medium) and passaged upon confluence. Plasmids VR1012, pElonginCΔ4, pCul5-HA, pCul5-myc, and pCul5ΔNedd8 have been described (19). pErbB2 and pCMV-HIF1-α HA were described previously (29, 33). Cells were transfected with Mirus LTI transfection reagent or Lipofectamine2000 (Invitrogen) according to manufacturers' instructions. The following antibodies used herein have been described previously (18); anti-HA antibody-agarose conjugate, anti-Elongin B, anti-Elongin C, anti-myc, anti-HA, and anti-human ribosomal P antigens. In addition, the following antibodies were used: anti-ErbB2 (Oncogene), anti-Cul5 (Santa-Cruz Biotechnology), anti-Cul2 (Zymed), anti-ubiquitin (Covance), anti-Hsp70, and anti-Hsp90 (Stressgen). Geldanamycin (Invivogen) was stored as a 1 mM stock solution in DMSO and used at the indicated concentrations. MG132 (EMD Biosciences) was stored as a 10 mM stock solution in DMSO and used at 5 μM unless otherwise indicated.

Immunoprecipitation and Western Blotting.

Immunoprecipitation was performed as described previously ( ). The eluted materials were then analyzed by SDS-PAGE and Western blotting as described (18).

RNA Interference.

siRNA duplexes (Dharmacon) were obtained as a smartpool. Duplexes were transfected according to Dharmacon's instructions using Lipofectamine2000 (Invitrogen). Cells were transfected at 50% confluence and harvested 72 h later.

shRNA Lentivirus Production and Transduction.

shRNA and lentivirus packaging system were obtained from The RNAi Consortium collection available through the Hit center at Johns Hopkins University as a gift from Jef Boeke. Lentiviruses were prepared, and cells were transduced following the protocol generated by the The RNAi Consortium at the Broad Institute. Briefly, 293T cells were plated in a 10-cm plate at a concentration of 1.3−1.5×10⁵ cells/mL and transfected at a confluence of approximately 70% with the following plasmids. Cul5 or GFP shRNA pLKO.1 (20 μg), pMD2G (6 μg), pRSV-Rev (5 μg), and pMDLg/pRRE (10 μg). Eighteen hours post transfection media was changed to high BSA virus production media. Virus was harvested 48 h post transfection. Supernatant was centrifuged to remove any cell debris. SKBR3 cells were infected by adding 10% of the harvested virus plus polybreene at a concentration of 8 μg/mL. Twenty-four hours post infection, media was replaced with fresh media containing puromycin at a concentration of 2 μg/mL. Cells were selected for 1 week before being used for the GA-induced degradation assay.

In Vivo Ubiquitination Assays.

293T cells were co-transfected with ErbB2, Cul5ΔNedd8, and control vector as indicated. At 48 h post transfection, the cells were treated with 5 μM MG132 for 18 h. ErbB2 was immunoprecipitated as described previously. Immunoprecipitates were washed with a 500 μM NaCl wash buffer. Samples were then analyzed by Western blot against ErbB2, ubiquitin, myc, and β-actin.

Immunofluorescent Staining.

293T cells were transfected with the indicated plasmids as described above. At 24 h post transfection, the cells were trypsinized and plated on glass coverslips at a dilution of one-eighth. Sixteen hours post-plating, cells were fixed in 4% paraformaldehyde, permeablized in 0.3% Triton X-100, then stained with the indicated antibodies. Cells were visualized, with a Zeiss Meta 510 confocal microscope and viewed with LSM software or a Nikon 90i and viewed with Velocity software as indicated.

### Example 1

### Cul5 Interacts with the Hsp90 Chaperone Complex

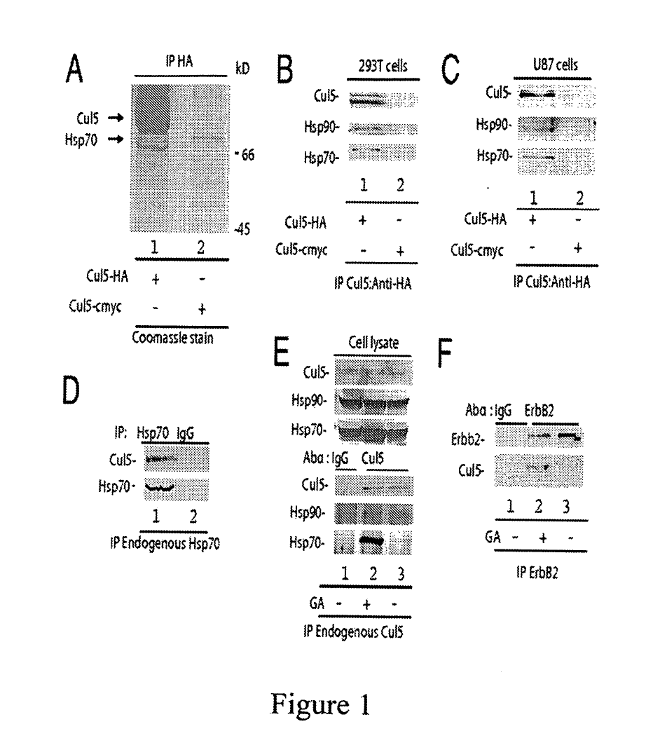

While the cellular function of Cul5 is poorly understood, it is known to be hijacked by the HIV type 1 (HIV-1) Vif protein to suppress the antiviral protein Apobec3G (A3G) (18-20) and by adenovirus E4orf6 and E1B55K to degrade p53, Mre 11, and DNA ligase IV (18, 21-25). To identify potential cellular partners of the Cul5 E3 ligase, HA-tagged Cul5 was immunoprecipitated and proteins that specifically co-immunoprecipitated with Cul5-HA but not with a cmyc-tagged Cul5 control protein were identified. In repeated experiments, Cul5-HA, but not Cul5-myc, co-precipitated Hsp70, as indicated, by mass spectrometry analysis (FIG. 1A). Interaction of Hsp70 with Cul5-HA but not Cul5-myc was confirmed by Western blotting with an anti-Hsp70 antibody (FIG. 1B). An interaction between Hsp90 and Cul5-HA was also observed (FIG. 1B), suggesting that Cul5 may be involved in the regulation of Hsp90 client proteins. Hsp90 is a chaperone that is required for the maturation and function of a number of classes of proteins, namely receptors, kinases, and transcription factors (7, 26). Hsp90 assembles with a number of co-chaperones, including Hsp70, and coordinates the maturation of its client proteins. Client protein maturation occurs via a dynamic ATP-dependent process (7). ATP hydrolysis or inhibition of Hsp90 function through drug treatment results in the reorganization of the chaperone complex and subsequent destabilization of the client protein (7). Interact ion of Cul5 with Hsp90 and Hsp70 was also confirmed in U87 cells (FIG. 1C). Immunoprecipitation of endogenous Hsp70 also co-precipitated endogenous Cul5 (FIG. 1D). Conversely, immunoprecipitation of endogenous Cul5 also co-precipitated endogenous Hsp90 and Hsp70 (FIG. 1E).

Hsp90 has been shown to be expressed at high levels, along with a number of its client proteins, in a variety of cancers and has therefore become a promising target for intervention (7). Treatment of cells with the benzoquinone ansamycin antibiotic geldanamycin (GA) or its analogs results in the proteasomal degradation of Hsp90 client proteins (16). GA is an ATPase inhibitor that binds the nucleotide-binding pocket with an affinity greater than that of ATP or ADP, shifting the chaperone complex into a conformation that favors client-protein degradation (27). ErbB2 is an Hsp90 client that has been extensively characterized. This receptor tyrosine kinase is overexpressed in approximately 30% of breast cancers and is required for tumor cell proliferation. Treatment of ErbB2-overexpressing cells with GA or its analogs results in rapid proteasomal degradation of ErbB2 (28, 29). While the U-box-containing E3 ligase, CHIP, has been implicated in the degradation of ErbB2 via Hsp/Hsc70, GA-mediated ErbB32 degradation still occurs in CHIP^{-/-} cells, suggesting that an additional E3 ligase is also involved in GA-mediated degradation of ErbB2 ( ).

To examine the possibility that Cul5, interacting with the Hsp90 chaperone complex, might serve as an E3 ligase to degrade Hsp90 client proteins, the interaction of Cul5 with ErbB2 was characterized. 293T cells transfected with ErbB2 or empty vector were treated with 2 μM GA or control DMSO and 5 μM MG132. Even though equal amounts of ErbB2 were immunoprecipitated, Cul5 interaction was detected only in GA-treated cells (FIG. 1F). These data demonstrate the endogenous interaction of Cul5 with ErbB2 and the Hsp90 chaperone complex, suggesting the involvement of Cul5 in the regulation of Hsp90 client proteins.

### Example 2

### Cul5 Co-Localizes with ErbB2 at the Plasma Membrane

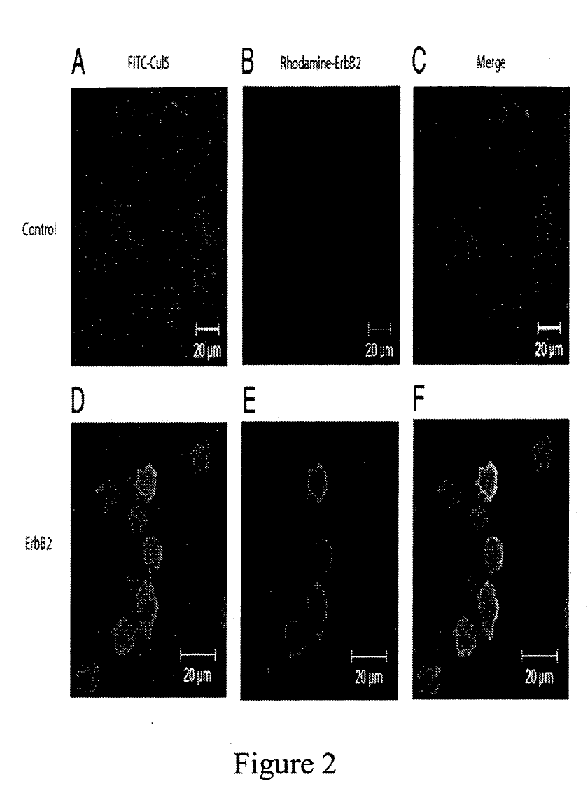

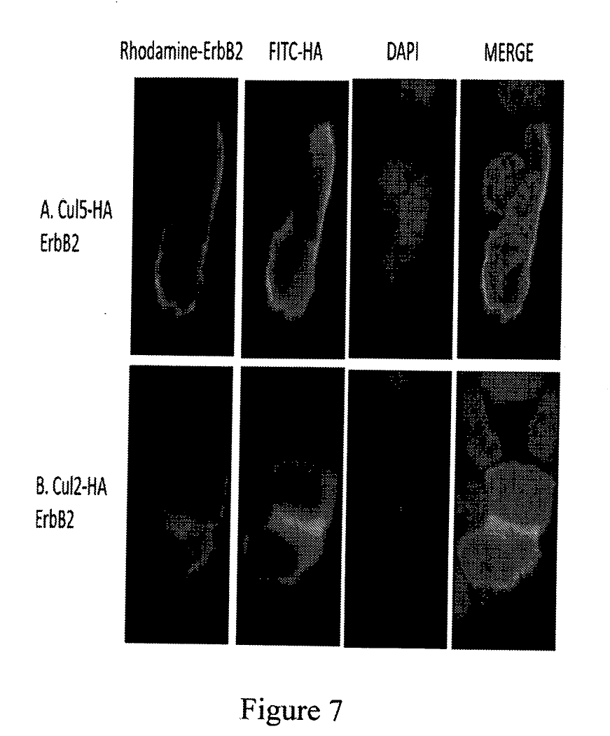

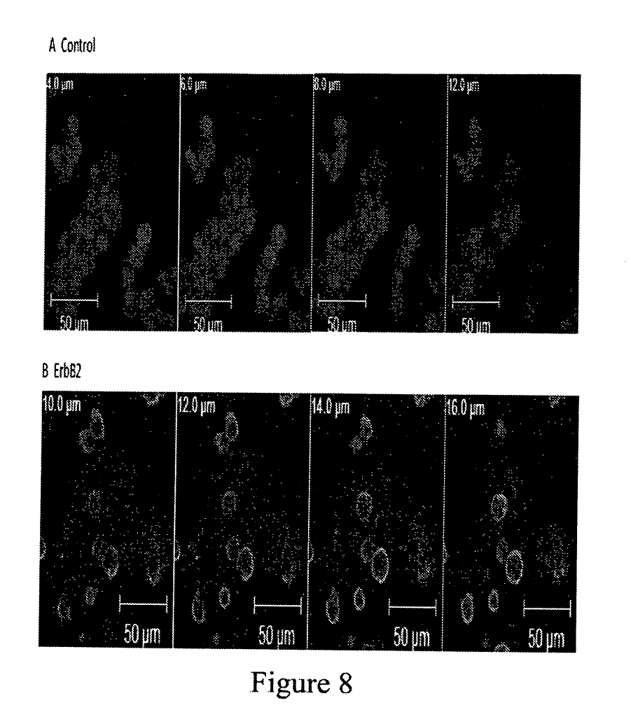

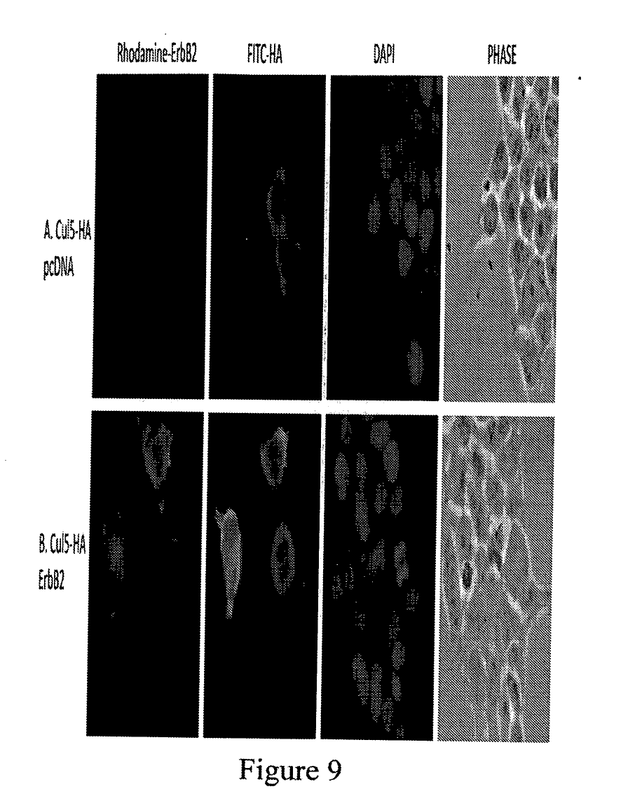

Because ErbB2 is a receptor tyrosine kinase, it was hypothesized that Cul5 might be acting on ErbB32 at the plasma membrane. 293T cells were therefore transfected with either the ErbB2 expression vector or empty vector control for 48 h, then stained for ErbB2 and endogenous Cul5. In the absence of ErbB2, Cul5 was evenly distributed throughout the cell (FIG. 2A). However, in the presence of ErbB2, Cul5 expression appeared to be concentrated at the plasma membrane (FIGS. 2D and E), suggesting that the presence of substrate results in the recruitment of Cul5 to the plasma membrane. This interaction was specific to Cul5, as Cul2 did not co-localize with ErbB2 (FIG. 7). Cul5 appears to co-localize with ErbB2 at the plasma membrane as seen in multiple 2-μm) frames of a z-stack (FIG. 8). ErbB2 expression did not affect cell integrity as seen by DAPI and phase contrast (FIG. 9).

### Example 3

### Cul5 is Required for Polyubiquitination and Proteasomal Degradation of ErbB2

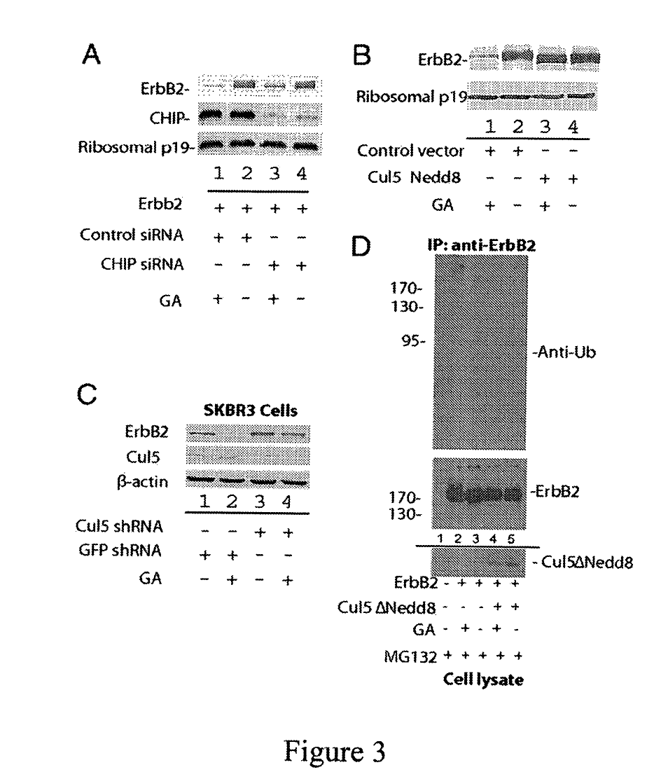

Treatment of ErbB2-expressing cells with GA is known to result in rapid proteasomal degradation of the receptor; however, degradation of ErbB2 still occurs in CHIP^{-/-} cells, suggesting that CHIP is necessary but not sufficient for GA-mediated degradation of ErbB2 ( ). In fact, the same phenotype was observed in the present system. Transfection with CHIP siRNA resulted in efficient CHIP knockdown; however, treatment with GA still resulted in ErbB2 degradation, supporting the hypothesis that an additional mechanism of GA-induced degradation is at work in this system (FIG. 3A).

To determine whether Cul5 is involved in ErbB2 degradation a dominant-negative mutant, Cul5ΔNedd8, was used to assess the requirement of Cul5 for ErbB2 degradation (FIG. 3B). In the absence of Cul5 Nedd8, treatment of ErbB2-transfected 293T cells with GA resulted in ErbB2 degradation (FIG. 3B, compare lanes 1 and 2). However, after the addition of Cul5ΔNedd8, ErbB2 was stabilized even in the presence of GA (FIG. 3B, compare lanes 3 and 1). It was also demonstrated that GA induced degradation of endogenous ErbB2 in the SKBR3 breast cancer cell line was inhibited when Cul5 was efficiently knocked-down. The stabilization of ErbB2 in the SKBR3 cells was observed even in the presence of GA when Cul5 expression was reduced by the shRNA strategy via a lentiviral delivery system (FIG. 3C, lane 4), compared to SKBR3 cells treated with control shRNA targeting GFP (FIG. 3C, lane 2). GA treatment induced polyubiquitination of ErbB2 (FIG. 3D, lane 2), when compared to the control treated cells (lane 3). Cul5ΔNedd8 also inhibited the polyubiquitination of ErbB2 induced by GA (FIG. 3D, compare lanes 2 and 4).

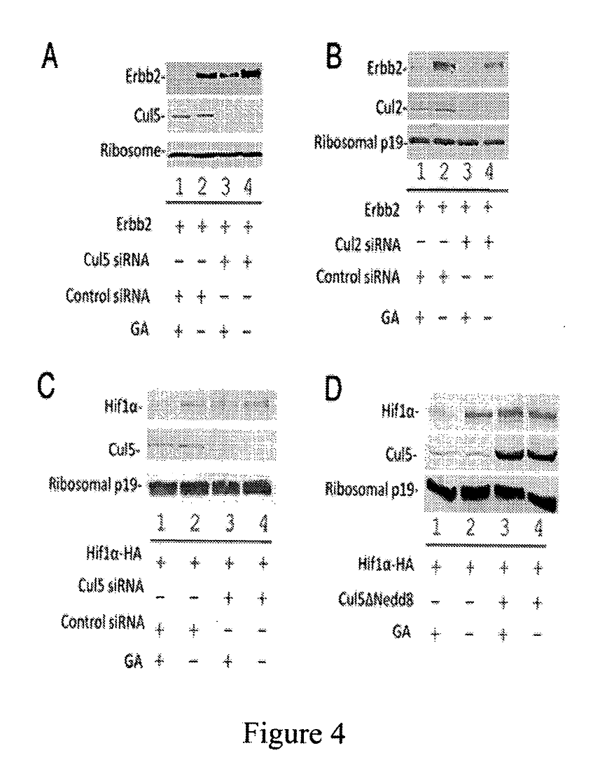

To further examine the role of Cul5 in GA-induced degradation of Hsp90 clients, siRNA was used against the Cul5 coding region to knock down Cul5 expression. Addition of Cul5-specific siRNA (FIG. 4A, lanes 3 and 4), but not control siRNA (FIG. 4A, lanes 1 and 2), resulted in efficient Cul5 knockdown. GA induced efficient degradation of ErbB2 in control siRNA-treated cells (FIG. 4A, compare lanes 1 and 2), but Cul5 knockdown served to stabilize ErbB2, even in the presence of GA (FIG. 4A, compare lanes 3 and 1). In contrast, knockdown of another Cullin E3 ligase (Cul2) had little effect on GA-induced ErbB2 degradation (FIG. 4B). Taken together, these data suggest that Cul5 is required for GA-induced polyubiquitination and degradation of the Hsp90 client protein ErbB2.

### Example 4

### Cul5 is Required for Degradation of HIF1-α, Another Hsp90 Client

These findings led to the hypothesis of whether Cul5 also participates in the regulation of other Hsp90 client proteins. HIF1-α is a transcription factor that is involved in the regulation of angiogenesis and glucose metabolism (30). Under normoxic conditions, the Cul2-VHL E3 ubiquitin ligase induces the degradation of HIF1-α, but under hypoxic conditions, HIF1-α is stabilized (31). Stabilization of HIF1-α under normoxic conditions has been observed in tumors and VHL-null or mutant cell lines (32). Constitutive expression of HIF1-α is important for vascularization, adaptation to hypoxia, and overall tumor survival. HIF1-α has also been shown to associate with Hsp90 and is sensitive to GA-induced degradation via a mechanism that is, independent of oxygen and VHL-Cul2 (7, 33). The potential involvement of Cul5 in GA-mediated, oxygen-independent degradation of HIF1-α was examined. In the presence of control siRNA, treatment with 3 μM GA for 16 h induced HIF1-α degradation (FIG. 4C, lane 1), when compared to control cells that were not subjected to GA (FIG. 4C, late 2). However, when Cul5 expression was reduced by siRNA against Cul5, GA-induced degradation of HIF1-α was inhibited (FIG. 4C, compare lane 3 to lane 1). A similar effect was observed with the Cul5 dominant negative mutant, Cul5ΔNedd8 (FIG. 4D). Thus, Cul5 apparently also plays a role in the regulation of the Hsp90 client protein HIF1-α, suggesting that Cul5 may regulate multiple Hsp90 client proteins.

### Example 5

### Cul5-Induced Degradation of ErbB2 Occurs Independent of ElonginB and ElonginC

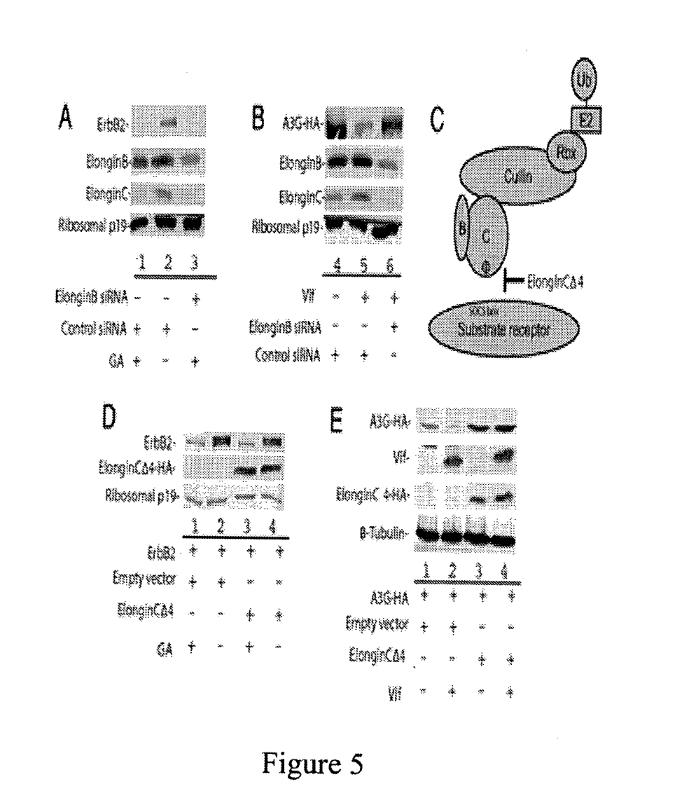

Cul5 classically requires the adaptor proteins ElonginB and ElonginC to efficiently induce the proteasomal degradation of its substrate. To determine whether ElonginB and ElonginC are required for ErbB2 degradation, siRNA was used against ElonginB. Interestingly, knockdown of ElonginB resulted in a significant decrease in ElonginC (FIGS. 5 A and B, lanes 3 and 6). This data supports the hypothesis that ElonginB may be involved in stabilization of ElonginC (34). In the presence of siRNA targeted against ElonginB, a defect in GA-induced degradation of ErbB2 was not observed when compared to nontargeting control siRNA (FIG. 5A, compare lanes 1 and 3). However, a defect in HIV Vif-mediated degradation of APOBEC3G was observed, suggesting an efficient decrease in ElonginB and ElonginC levels (FIG. 5B, compare lanes 2 and 3). To further evaluate the role of the ElonginB-C adaptor module in Cul5-mediated ErbB2 degradation, an ElonginC dominant negative mutant was used. This mutant can bind Cul5 but can no longer bind the SOCS box in the substrate receptor (FIG. 5C). This has been observed for both HIV-1 Vif and adenovirus E4orf6 (19, 22). Transfection of the ElonginC dominant, negative mutant that can no longer interact with the SOCS box had no effect on GA-mediated ErbB2 degradation (FIG. 5D, compare lane 1 with lane 3) but did effect Vif-mediated A3G degradation (FIG. 5E, compare lanes 2 and 4). Because this mutant can no longer interact with the SOCS box, this data suggests that Cul5-mediated degradation of ErbB2 occurs in the absence of a SOCS box containing substrate receptor. In addition, this data suggests Cul5-mediated ErbB2 regulation occurs via an ElonginB-ElonginC-independent, E3 ubiquitin ligase.

### REFERENCES

1. Dai C, Whitesell L. Rogers A B, Lindquist S (2007) Heat shock factor 1 is a powerful multifaceted modifier of carcinogenesis. Cell 130:1005-1018.
2. Solit D B, Rosen N (2006) Hsp90: A novel target for cancer therapy. Curr Top Med Chem 6:1205-1214.
3. Wegele H, Muller L, Buchner J (2004) Hsp70 and Hsp90—a relay team for protein folding. Rev Physiol Biochem Pharmacol 151:1-44.
4. Zhang H, Burrows F (2004) Targeting multiple signal transduction pathways through inhibition of Hsp90. J Mol Med 82:488-499.
5. Waterston R H. et al. (2002) Initial sequencing and comparative analysis of the mouse genome. Nature 420:520-562.
6. Citri A, Kochupurakkal B S, Yarden Y (2004) The achilles heel of ErbB-2/HER2: Regulation by the Hsp90 chaperone machine and potential for pharmacological intervention. Cell Cycle 3:51-60.
7. Neckers L (2002) Hsp90 inhibitors as novel cancer chemotherapeutic agents. Trends Mol Med 8 (4 Suppl):S55-S61.
8. Petroski M D, Deshaies R J (2005) Function and regulation of cullin-RING ubiquitin ligases. Nat Rev Mol Cell Biol 6:9-20.
9. Pintard L, Willems A, Peter M (204) Cullin-based ubiquitin ligases: Cul3-BTB complexes join the family. EMBO J 23; 1681-1687.
10. Deshaies R J (1999) SCF and Cullin/Ring H2-based ubiquitin ligases. Annu Rev Cell Dev Biol 15:435-467.
11. Kamura T. et al. (2004) VHL-box and SOCS-box domains determine binding specificity for Cul2-Rbx1 and Cul5-Rbx2 modules of ubiquitin ligases. Genes Dev 18:3055-3065.
12. Mehle A, Thomas E R, Rajendran K S, Gabuzda D (2006) A zinc-binding region in Vif binds Cul5 and determines Cullin selection. J Biol Chem 281: 17259-17265.
13. Xiao Z, Ehrlich E, Luo K, Xiong Y, Yu X F (2007) Zinc chelation inhibits HIV Vif activity and liberates antiviral function of the cytidine deaminase APOBEC3G. Faseb J 21:217-222.
14. Xiao. Z, et al. (2007) Characterization of a novel Cullin 5 binding domain in HIV-1. Vif. J Mol Biol 373:541-550.
15. Xiao Z, et al. (2006) Assembly of HIV-1 Vif-Cul5 E3 ubiquitin ligase through a novel zinc-binding domain-stabilized hydrophobic interface in Vif. Virology 349:290-299.
16. Blagosklonny M V (2002) Hsp-90-associated oncoproteins: Multiple targets of geldanamycin and its analogs. Leukemia 16:455-462.
17. Fay M J, et al. (2003) Analysis of CUL-5 expression in breast epithelial cells, breast cancer cell lines, normal tissues and tumor tissues. Mol Cancer 2:40.
18. Yu X, et al. (2003) Induction of APOBEC3G ubiquitination and degradation by an HIV-1 Vif-Cul5-SCF complex. Science 302:1056-1060.
19. Yu Y, Xiao Z, Ehrlich E S, Yu X, Yu X F (2004) Selective assembly of HIV-1 Vif-Cul5-ElonginB-ElonginC E3 ubiquitin ligase complex through a novel SOCS box and upstream cysteines. Genes Dev 18:2867-2872.
20. Mehle A. Goncalves J, Santa-Marta M, McPike M, Gabuzda D (2004) Phosphorylation of a novel SOCS-box regulates assembly of the HIV-1 Vif-Cul5 complex that promotes APOBEC3G degradation. Genes Dev 18:2861-2866.
21. Stracker T H, Carson C T, Weitzman M D (2002) Adenovirus oncoproteins inactivate the Mre11-Rad50-NBS1 DNA repair complex. Nature 418:348-352.
22. Luo K, et al. (2007) Adenovirus E4orf6 assembles with Cullin5-ElonginBElonginC E3 ubiquitin ligase through an HIV/SIV Vif-like BC-box to regulate p53. Faseb J 21:1742-1750.
23. Harada J N, Shevchenko A, Shevchenko A, Pallas D C, Berk A J (2002) Analysis of the adenovirus E1B-55K-anchored proteome reveals its link to ubiquitination machinery. J Virol 76:9194-9206.
24. Querido E, et al (2001) Degradation of p53 by adenovirus E4orf6 and E1B55K proteins occurs via a novel mechanism involving a Cullin-containing complex. Genes Dev 15:3104-3117.
25. Baker A, Rohleder K J, Hanakahi L A, Ketner G (2007) Adenovirus E4 34k and E1b 55k oncoproteins target host DNA ligase IV for proteasomal degradation. J Virol 81:7034-7040.
26, Young J C, Agashe V R, Siegers K, Hartl F U (2004). Pathways of chaperone mediated protein folding in the cytosol. Nat Rev Mol Cell Biol 5:781-791.
27. Prodromou C, et al. (1997) Identification and structural characterization of the ATP/ADP-binding site in the Hsp90 molecular chaperone. Cell 90:65-75.
28. Zhou P, et al. (2003) ErbB2 degradation mediated by the co-chaperone protein CHIP. J Biol Chem 278:13829-13837.
29. Xu W, et al (2002) Chaperone-dependent E3 ubiquitin ligase CHIP mediates a degradative pathway for c-ErbB2/Neu. Proc Natl Acad Sci USA 99:12847-12852.
30. Safran M, Kaelin W G, Jr (2003) HIF hydroxylation and the mammalian oxygen-sensing pathway. J Clin Invest 111:779-783;
31. Lonergan K M, et al. (1998) Regulation of hypoxia-inducible mRNAs by the von Hippel-Lindau tumor suppressor protein requires binding to complexes containing elongins B/C and Cul2. Mol Cell Biol 183:732-741.
32. Maxwell P H, et al. (1999) The tumour suppressor protein VHL targets hypoxia inducible factors for oxygen-dependent proteolysis. Nature 399:271-275.
33. Isaacs J S, Jung Y J, Neekers I. (2004) Aryl hydrocarbon nuclear translocator (ARNT) promotes oxygen-independent stabilization of hypoxia-inducible factor-1alpha by modulating an Hsp90-dependent regulatory pathway. J Biol Chem 279:16128-16135.
34. Stebbins C E, Kaelin W G, Jr, Pavletich N P (1999) Structure of the VHLElonginC-ElonginB complex: Implications for VHL tumor suppressor function. Science 284:455-461.
35. Chen X P, et al. (2002) Pim serine/threonine kinases regulate the stability of Socs-1 protein. Proc Natl Acad Sci USA 99:2175-2180.

We claim:

1. A method for determining whether an agent causes an increase in the expression of Cullin 5 protein comprising the steps of:
(a) contacting the agent with a eukaryotic cell under conditions which, in the absence of the agent, permit expression of Cullin 5 protein;
(b) determining, after a suitable period of time, the amount of expression of Cullin 5 protein in the cell; and
(c) comparing the amount of expression determined in step (b) with the amount of expression which occurs in the absence of the agent, whereby an increased amount of expression in the presence of the agent indicates that the agent causes an increase in the expression of Cullin 5 protein.

2. The method of claim 1, wherein the cell is present in a cell culture.

3. The method of claim 1, wherein the cell is a tumor cell.

4. The method of claim 1, wherein determining the amount of expression of Cullin 5 protein in the cell is performed by determining the amount of Cullin 5 protein-encoding mRNA present in the cell.

5. The method of claim 1, wherein determining the amount of expression of Cullin 5 protein in the cell is performed by determining the amount of Cullin 5 protein present in the cell.

6. The method of claim 5, wherein determining the amount of Cullin 5 protein in the cell is performed using an antibody specific for such protein.

7. A method for determining whether an agent causes an increase in the activity of Cullin 5 protein comprising the steps of:
(a) contacting the agent with a eukaryotic cell under conditions which, in the absence of the agent, permit activity of Cullin 5 protein;
(b) determining, after a suitable period of time, the amount of activity of Cullin 5 protein in the cell; and
(c) comparing the amount of activity determined in step (b) with the amount of activity of Cullin 5 protein which occurs in the absence of the agent, whereby an increased amount of activity in the presence of the agent indicates that the agent causes an increase in the activity of Cullin 5 protein.

8. The method of claim 7, wherein the cell is present in a cell culture.

9. The method of claim 7, wherein the cell is a tumor cell.

10. A method for increasing the expression of Cullin 5 in a eukaryotic cell comprising introducing into the cell an agent, which specifically increases the expression of Cullin 5 protein in the cell.

11. The method of claim 10, wherein the agent, is a polynucleotide.

12. The method of claim 11, wherein the polynucleotide is an expression vector encoding Cullin 5.

13. The method of claim 10, wherein the cell is a tumor cell.

14. A method for treating a subject afflicted with an HSP90 client protein related disorder comprising administering to the subject a therapeutically effective amount of an agent which specifically increases the expression of Cullin 5 protein in the cells of the subject.

15. The method of claim 14, further comprising the step of administering an HSP90 inhibitor to the subject.

16. The method of claim 14, wherein the subject is human.

17. The method of claim 14, wherein the agent is a polynucleotide.

18. The method of claim 17, wherein the polynucleotide is an expression vector encoding Cullin 5.

19. The method of claim 14, wherein the HSP client protein is selected from the group consisting of ErbB2, Hif-1α, c-Rav-1, Akt/PKB, CDK4, Polo-1 kinase, Met, mutant p53, and hTERT.

20. The method of claim 14, wherein the HSP client protein is ErbB2.

21. The method of claim 14, wherein the HSP client protein is Hif-1α.
